(19) United States
(12) Patent Application Publication
(10) Pub. No.: US 2026/0131038 A1
(43) Pub. Date: May 14, 2026

(54) TEST DEVICE, STERILIZATION MONITORING SYSTEM AND METHOD

(71) Applicant: Solventum Intellectual Properties Company, Maplewood, MN (US)

(72) Inventors: Wensheng Xia, Woodbury, MN (US); G. Marco Bommarito, Stillwater, MN (US); Naiyong Jing, St. Paul, MN (US)

(21) Appl. No.: 19/117,126

(22) PCT Filed: Sep. 13, 2023
(86) PCT No.: PCT/IB2023/059088

§ 371 Date: Mar. 31, 2025

Related U.S. Application Data
(60) Provisional application No. 63/378,541, filed on Oct. 6, 2022.

(51) Int. Cl. A61L 2/28; A61L 2/07; A61L 103/15; G01N 27/02

(52) U.S. Cl. CPC A61L 2/28; A61L 2/07; G01N 27/028; A61L 2103/15; A61L 2202/122; A61L 2202/14

(57) ABSTRACT

A test device for monitoring sterilization using a steam sterilant in a chamber is provided. The test device includes a test stack. The test stack includes an entrance layer including an entrance hole. The test stack further includes a sensor layer spaced apart from the entrance layer. The sensor layer includes at least one pair of electrodes disposed on the sensor layer. The test stack further includes at least one sensor coating disposed on at least one portion of the sensor layer and including an electrically active polymer. The test stack further includes an intermediate layer disposed between the entrance layer and the sensor layer. The intermediate layer is disposed in fluidically connects the entrance hole and the at least one sensor coating. The intermediate layer is configured to allow a flow of the steam sterilant received from the entrance hole to the at least one sensor coating.

### TECHNICAL FIELD

The present disclosure relates generally to sterilization, and more particularly, relates to a test device for monitoring sterilization, a sterilization monitoring device including the test device, and a method for monitoring sterilization in a chamber.

### BACKGROUND

Sterilization of medical and hospital equipment may not be effective until a steam sterilant has been in contact with all surfaces of materials being sterilized in a proper combination of time, temperature, and steam quality. In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing stage, in which a load (i.e., the articles being sterilized) is subjected to steam under pressure for a recognized, predetermined combination of time and temperature to effect sterilization. The third phase is a drying phase in which condensation formed during the first two phases is removed by evacuating the chamber.

Any air that is not removed from the sterilizer during the air removal phase of the cycle or which leaks into the sterilizer during a sub atmospheric pressure stage due to, for example, faulty gaskets, valves or seals, may form air pockets within any porous materials present. Such air pockets may create a barrier to steam penetration, thereby preventing adequate sterilizing conditions being achieved for all surfaces of the load during the sterilizing phase. For example, these air pockets may prevent the steam from reaching interior layers of materials, such as hospital linens or fabrics. In some other examples, these air pockets may prevent the steam from penetrating hollow spaces of tubes, catheters, syringe needles, and the like. Further, non-condensable gas (generally air) present within the sterilizer is a poor sterilant and may decrease sterilization efficacy. A percentage of non-condensable gas in the steam should be less than or equal to 3.5% by volume. Therefore, the presence of air pockets and/or non-condensable gas may affect a steam quality of the steam sterilant. As a result, proper sterilization may not occur due to reduced steam quality. A few more factors that may affect steam quality include insufficient steam supply, water quality, degassing, design of the sterilizer chamber, etc.

### SUMMARY

In a first aspect, the present disclosure provides a test device for monitoring sterilization using a steam sterilant in a chamber. The test device includes a test stack defining a major plane and a perimeter. The test stack includes an entrance layer including an entrance hole extending through the entrance layer. The entrance hole is in fluidic connection with the chamber. The test stack further includes a sensor layer spaced apart from the entrance layer. The sensor layer includes at least one pair of electrodes disposed on the sensor layer. The test stack further includes at least one sensor coating disposed on at least one portion of the sensor layer and including an electrically active polymer. The at least one sensor coating is spaced apart from the entrance hole at least along the major plane of the test stack. The at least one sensor coating is electrically coupled to the at least one pair of electrodes. The test stack further includes an intermediate layer disposed between the entrance layer and the sensor layer. The intermediate layer fluidically connects the entrance hole and the at least one sensor coating. The intermediate layer is configured to allow a flow of the steam sterilant received from the entrance hole to the at least one sensor coating. The at least one sensor coating is configured to change an electrical impedance across the at least one pair of electrodes upon contact of the steam sterilant with the at least one sensor coating.

In a second aspect, the present disclosure provides a sterilization monitoring system including the test device of the first aspect. The sterilization monitoring system further includes a reader configured to at least partially receive the test device therein for measuring the electrical impedance across the at least one pair of electrodes.

In a third aspect, the present disclosure provides a sterilization system including the sterilization monitoring system of the second aspect. The sterilization system further includes a sterilizer including a chamber configured to receive the test device therein. The sterilizer is configured to perform a sterilization process on the test device using a steam sterilant within the chamber.

In a fourth aspect, the present disclosure provides a method for monitoring sterilization in a chamber using the test device of first aspect. The method includes disposing the test device within the chamber. The method further includes performing a sterilization process on the test device using a steam sterilant. The method further includes removing the test device from the chamber. The method further includes at least partially inserting the test device within a reader for measuring the electrical impedance across the at least one pair of electrodes.

In a fifth aspect, the present disclosure provides a test device for monitoring sterilization using a steam sterilant in a chamber. The test device includes a test stack defining a major plane and a perimeter. The test stack includes a top layer including a first major surface proximal to the chamber, a second major surface opposite to the first major surface, and an entrance hole extending from the first major surface at least partially through the top layer and disposed in fluidic connection with the chamber. The top layer incorporates at least one intermediate path at least partially aligned with and disposed in fluidic connection with the entrance hole. The at least one intermediate path defines a path length along the major plane and a path depth normal to the major plane. The at least one intermediate path extends from the second major surface at least partially through the top layer along the path depth. The at least one intermediate path is spaced apart from the perimeter of the test stack. The test stack further includes a sensor layer disposed adjacent to the second major surface of the top layer. The sensor layer includes at least one pair of electrodes disposed on the sensor layer. The test stack further includes at least one sensor coating disposed on at least one portion of the sensor layer and including an electrically active polymer. The at least one intermediate path of the top layer extends from the entrance hole to the at least one sensor coating at least along the path length, such that the at least one intermediate path fluidically connects the entrance hole with the at least one sensor coating. The at least one sensor coating is electrically coupled to the at least one pair of electrodes on the sensor layer. The top layer is configured to allow a flow of the steam sterilant from the entrance hole to the at least one sensor coating. The at least one sensor coating is configured to change an electrical impedance across the pair of electrodes upon contact of the steam sterilant with the at least one sensor coating.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a block diagram of a sterilization system, according to an embodiment of the present disclosure;

FIG. 2 is a perspective top view of a test device of the sterilization system of FIG. 1, according to an embodiment of the present disclosure;

FIG. 3 is a sectional side view of the test device of FIG. 2 comprising a test stack taken along a line A-A′ as shown in FIG. 2, according to an embodiment of the present disclosure;

FIG. 4 is a top view of the test stack of FIG. 3, according to an embodiment of the present disclosure;

FIG. 5 is a bottom view of the test stack of FIG. 3, with some layers not shown, according to an embodiment of the present disclosure;

FIG. 6 schematically shows a reader of the sterilization system of FIG. 1, according to an embodiment of the present disclosure;

FIG. 7 is a bottom view of a test device, with some layers not shown, according to another embodiment of the present disclosure;

FIG. 8 is a sectional side view of a test device, according to another embodiment of the present disclosure;

FIG. 9 is a bottom view of the test device of FIG. 8, with some layers not shown;

FIG. 10 is a sectional side view of a test device, according to another embodiment of the present disclosure;

FIG. 11 is a bottom view of the test device of FIG. 10, with some layers not shown;

FIG. 12 is a bottom view of a test device, with some layers not shown, according to another embodiment of the present disclosure;

FIG. 13 is a bottom view of a test device, with some layers not shown, according to another embodiment of the present disclosure;

FIG. 14 is a bottom view of a test device, with some layers not shown, according to another embodiment of the present disclosure;

FIG. 15 is a bottom view of a test device, with some layers not shown, according to another embodiment of the present disclosure;

FIG. 16 is a sectional side view of a test device, according to another embodiment of the present disclosure; and

FIG. 17 is a flowchart for a method for monitoring sterilization in a chamber using the test device of FIG. 2, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

Steam sterilizers are widely used in medical centers and hospitals to sterilize medical equipment. Frequent testing or monitoring of steam quality may be essential to ensure a safe use of the medical equipment in a medical treatment. In other words, regular testing may have to be conducted to check effectiveness of air removal during air removal phase of the sterilization process, prior to subjecting the steam to a given load (i.e., medical equipment). One of the ways to monitor steam quality of the steam sterilant is a Bowie-Dick test. In general, the Bowie-Dick test uses an indicator sheet and a test pack having stack of freshly laundered towels. In some cases, the indicator sheet is a chemical indicator sheet. In some cases, the indicator sheet is a bio indicator sheet. In some cases, the test pack used in the Bowie-Dick test includes a disposable test pack.

Although conventional technique of conducting the Bowie-Dick test by using the test pack is generally recognized as an adequate procedure for determining the steam quality of the steam sterilant or efficacy of the air removal stage of steam sterilization process, it may face some challenges. A uniform change in color of the indicator sheet indicates that all air was removed and replaced by steam. In some cases, an operator may not accurately interpret a change in color of the indicator sheet, and this may further lead to erroneous classification of test results. Therefore, by using the test pack, the Bowie-Dick test may not always provide accurate test results due to possibility of human intervention errors while analyzing the test pack and/or the indicator sheet.

Further, to maintain a record/logbook of Bowie-Dick test results of one or more sterilizers, the operator may have to do a lot of scanning of the image of test packs, photocopying the test results, and manually recording the test results. It may be time consuming for the operator to manually maintain the logbook of the Bowie-Dick test results. As a result, throughput of a steam sterilizer may be reduced due to manual recording of the test results. Therefore, while using the test packs for conducting the Bowie-Dick tests, regularly updating the logbook of the Bowie-Dick test results may be difficult, erroneous, and time consuming. Moreover, for maintaining the logbook of the Bowie-Dick test results, a large quantity of paper may also be wasted on a regular basis.

The present disclosure relates to a test device for monitoring sterilization using a steam sterilant in a chamber. The test device includes a test stack defining a major plane and a perimeter. The test stack includes an entrance layer including an entrance hole extending through the entrance layer. The entrance hole is in fluidic connection with the chamber. The test stack further includes a sensor layer spaced apart from the entrance layer. The sensor layer includes at least one pair of electrodes disposed on the sensor layer. The test stack further includes at least one sensor coating disposed on at least one portion of the sensor layer and including an electrically active polymer. The at least one sensor coating is spaced apart from the entrance hole at least along the major plane of the test stack. The at least one sensor coating is electrically coupled to the at least one pair of electrodes. The test stack further includes an intermediate layer disposed between the entrance layer and the sensor layer. The intermediate layer fluidically connects the entrance hole and the at least one sensor coating. The intermediate layer is configured to allow a flow of the steam sterilant received from the entrance hole to the at least one sensor coating. The at least one sensor coating is configured to change an electrical impedance across the at least one pair of electrodes upon contact of the steam sterilant with the at least one sensor coating.

The present disclosure also provides a sterilization system including a sterilizer. The sterilizer includes a chamber configured to receive the test device. The sterilizer is configured to perform a sterilization process on the test device using the steam sterilant within the chamber.

For monitoring sterilization using the steam sterilant, the test device is placed within the chamber of the sterilizer and the sterilization process is initiated. As the intermediate layer fluidically connects the entrance hole with the at least one sensor coating, and the entrance hole is in fluidic connection with the chamber, the chamber is in indirect fluidic connection with the at least one sensor coating.

In the presence of any non-condensable gas or air within the chamber, air may contact the at least one sensor coating via the intermediate layer, and this may prevent the steam sterilant to make any contact with the at least one sensor coating. Hence, during a real-time sterilization process, in the presence of air, the steam sterilant may not reach hollow spaces and interior pockets of medical equipment subjected to sterilization. In the absence of the non-condensable gas or air within the chamber, the steam sterilant may be able to contact the at least one sensor coating via the intermediate layer. Hence, in the absence of air, the steam sterilant may reach hollow spaces and interior pockets of the medical equipment subjected to sterilization. In other words, for an acceptable quality of the steam sterilant, there should not be any presence of air within the chamber of the sterilizer.

In some embodiments, the intermediate layer includes a permeable material. The permeability of the permeable material of the intermediate layer is configured to allow the flow of the steam sterilant through the intermediate layer in order to fluidically connect the entrance hole with the at least one sensor coating. The permeability of the permeable material of the intermediate layer may offer a considerable resistance to the flow of the steam sterilant through the intermediate layer. Particularly, the permeable material of the intermediate layer may provide the resistance that corresponds to a resistance provided by different routes and passages that the steam sterilant may have to follow to reach the hollow spaces and interior pockets of the medical equipment in a real-time sterilization process. The resistance provided by the permeable material of the intermediate layer may therefore represent the resistance of various flow channels leading to hidden spaces of tubes, catheters, syringe needles, and the like. The resistance provided by the permeable material of the intermediate layer to the flow of the steam sterilant may depend on various properties of the permeable material.

In some other embodiments, the intermediate layer further includes at least one internal channel defining a channel length along the major plane and a channel depth normal to the major plane. The at least one internal channel is spaced apart from the perimeter of the test stack. The at least one internal channel extends through the intermediate layer along the channel depth. The at least one internal channel extends from the entrance hole to the at least one sensor coating at least along the channel length, such that the at least one internal channel fluidically connects the entrance hole with the at least one sensor coating.

The at least one internal channel may offer a considerable resistance to the flow of the steam sterilant through the at least one internal channel. Particularly, the resistance provided by the at least one internal channel may correspond to the resistance provided by the different routes and the passages that the steam sterilant may have to follow to reach the hollow spaces and interior pockets of the medical equipment in a real-time sterilization process. The resistance provided by the at least one internal channel to the flow of the steam sterilant may depend on a shape and dimensions of the at least one internal channel. Moreover, the shape and the dimensions of the at least one internal channel may vary based on different application attributes.

Further, upon contact with the steam sterilant, the at least one sensor coating is further configured to change the electrical impedance across the at least one pair of electrodes beyond a predetermined threshold impedance. The predetermined threshold impedance may be selected based on various application attributes. Therefore, upon contact of the steam sterilant with the at least one sensor coating, the electrical impedance across the at least one pair of electrodes is beyond the predetermined threshold impedance. Further, in the presence of air, the steam sterilant may not contact the at least one sensor coating, and the electrical impedance across the at least one pair of electrodes is below the predetermined threshold impedance.

The present disclosure further provides a sterilization monitoring system including the test device and a reader configured to at least partially receive the test device therein for measuring the electrical impedance across the at least one pair of electrodes. The sterilization monitoring system is a part of the sterilization system of the present disclosure. Further, the entrance layer and the intermediate layer of the test device at least partially define a cutout disposed at the perimeter of the test stack. Each of the at least one pair of electrodes at least partially extends into the cutout. The cutout is configured to at least partially receive one or more terminals of the reader therein for measuring the electrical impedance across the at least one pair of electrodes. A magnitude of the electrical impedance across the at least one pair of electrodes indicates the presence or absence of air in the sterilizer and the steam quality of the steam sterilant. The reader provides a pass result upon determining that the electrical impedance across the at least one pair of electrodes is beyond the predetermined threshold impedance. Further, the reader provides a fail result upon determining that the electrical impedance across the at least one pair of electrodes is below the predetermined threshold impedance. Therefore, the reader may provide an accurate pass or fail result of a steam sterilization process based on a comparison between the predetermined threshold impedance and the electrical impedance across the at least one pair of electrodes.

In cases where the electrical impedance across the at least one pair of electrodes is beyond the predetermined threshold impedance, the operator may also determine a quantitative relevancy of the pass result based on a magnitude of a difference between the electrical impedance and the predetermined threshold impedance. In cases where the electrical impedance across the at least one pair of electrodes is not beyond the predetermined threshold impedance, the operator may also determine a quantitative relevancy of the fail result based on the magnitude of the difference between the electrical impedance and the predetermined threshold impedance.

Further, the test device is a built-in and a stand-alone unit which can be used with any sterilizer. In contrast to the conventional technique of monitoring sterilization by using the test pack and/or indicator sheets, and then manually interpreting the change in color of the indicator sheets, the sterilization monitoring system including the test device may require minimal human interpretation to determine the pass/fail result of the sterilization process. Therefore, the test device and the sterilization monitoring system of the present disclosure may provide an accurate classification of test results that could have been otherwise erroneous due to possible human intervention errors. Moreover, as the test device is being used here for monitoring the steam quality of the steam sterilant by measuring the electrical impedance across the at least one pair of electrodes, the test device of the present disclosure may be called as an electronic testing unit or an electronic test card. In some cases, the sterilization monitoring system including the test device and the reader may also provide a digital pass/fail result of the steam quality of the steam sterilant.

In contrast to the conventional techniques for monitoring sterilization, the sterilization monitoring system of the present disclosure may eliminate a need for scanning of images of the test packs (indicator sheets), photocopying the test results, and manually recording the test results. Moreover, the sterilization monitoring system including the test device may eliminate the need to maintain a record/logbook of Bowie-Dick test results of one or more sterilizers. This may also reduce a possibility of misplacing the various test results of the steam quality of the steam sterilant. Therefore, an overall throughput of the sterilizer may be increased due to minimal manual recording and/or manual maintenance of the test results. The sterilization monitoring system may allow a faster and an easier testing process for the operator to accurately monitor the steam quality of the steam sterilant and/or validate proper air removal in the chamber of the sterilizer. Consequently, the disclosed sterilization monitoring system may increase an efficiency of the sterilizer and decrease a complexity of the process to monitor the steam quality of the steam sterilant. Moreover, the sterilization monitoring system may also save a large amount of paper that was otherwise wasted in the conventional techniques for monitoring sterilization.

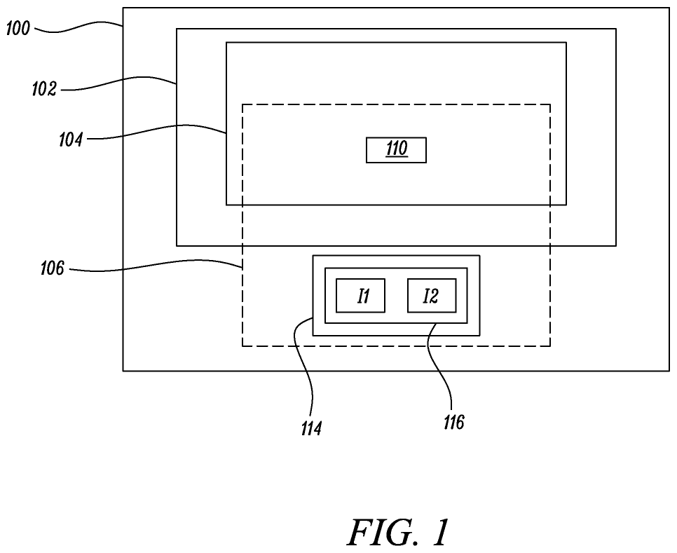

Referring now to Figures, FIG. 1 illustrates a block diagram of a sterilization system 100. The sterilization system 100 includes a sterilizer 102 including a chamber 104. The chamber 104 may have one or more environmental conditions. In some cases, the environmental condition may be related to conditions inside the chamber 104, and may include time, sterilant, temperature, pressure, or combinations thereof. In some embodiments, the chamber 104 may be made of various materials such as, but not limited to, steel, metal, polymer, or any other materials. The chamber 104 is configured to receive a steam sterilant therein. When steam is used as the steam sterilant, an object of a sterilization process is to bring steam at an appropriate temperature into contact with all surfaces of the articles being sterilized for an appropriate period of time.

The sterilization system 100 further includes a sterilization monitoring system 106. The sterilization monitoring system 106 includes a test device 110 for monitoring sterilization using the steam sterilant in the chamber 104. The chamber 104 is configured to receive the test device 110 therein. The sterilizer 102 is configured to perform the sterilization process on the test device 110 using the steam sterilant within the chamber 104.

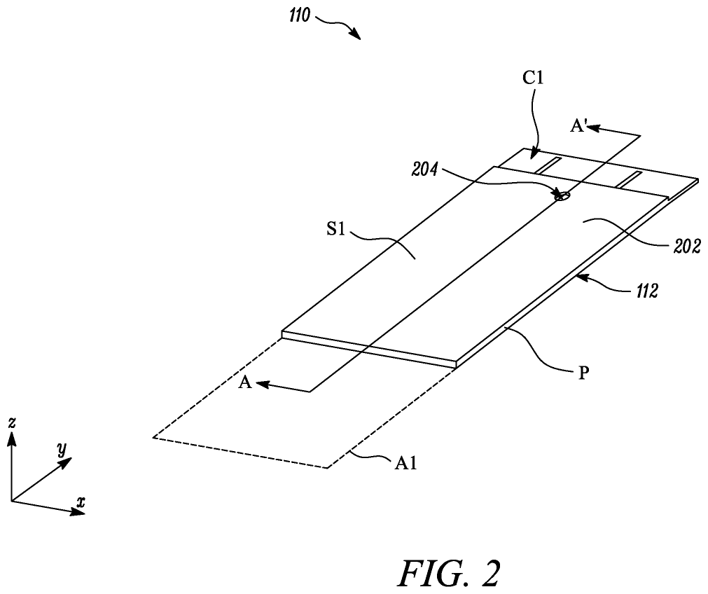

FIG. 2 is a perspective top view of the test device 110, according to an embodiment of the present disclosure. The test device 110 defines mutually orthogonal x, y, and z-axes. The test device 110 includes a test stack 112 defining a major plane A1 and a perimeter P. The x and y-axes are in-plane axes of the test stack 112, while the z-axis is a transverse axis disposed along a thickness of the test stack 112. In other words, the x and y-axes are disposed along the major plane A1 of the test stack 112, while the z-axis is perpendicular to the major plane A1 of the test stack 112. The major plane A1 therefore corresponds to the x-y plane.

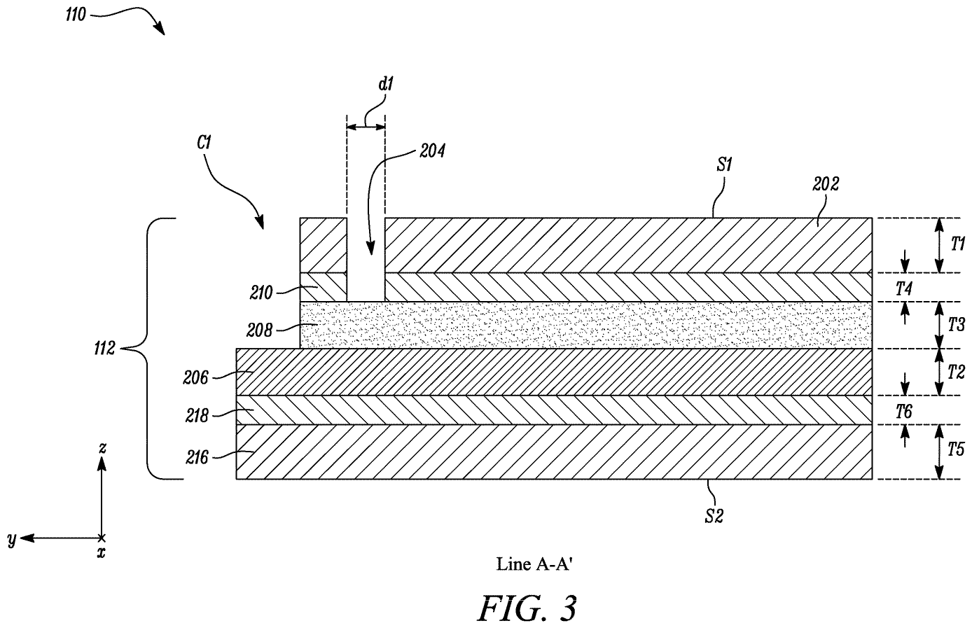

FIG. 3 is a sectional side view of the test device 110 comprising the test stack 112 taken along a line A-A′ as shown in FIG. 2, according to an embodiment of the present disclosure. The test stack 112 includes an entrance layer 202 including an entrance hole 204 extending through the entrance layer 202. In some embodiments, the entrance layer 202 includes polyethylene terephthalate (PET). Further, in some embodiments, the entrance layer 202 may be made of a metallic layer such as aluminum foil, a polymeric layer such as polyurethane or a polyester layer, without any limitations. The entrance layer 202 defines a thickness T1 along the z-axis. In some cases, the thickness T1 of the entrance layer is about 10 mil. The entrance layer 202 at least partially forms an external surface S1 of the test stack 112. In some embodiments, the test stack 112 may also include a graphics layer (not shown) at least partially forming the external surface S1 of the test stack 112. The graphics layer may include labeling, product logo, product specifications, and the like.

The entrance hole 204 is in fluidic connection with the chamber 104 (shown in FIG. 1). In the illustrated embodiment of FIG. 3, the entrance hole 204 is circular and, therefore, has a diameter d1. In some other embodiments, the entrance hole 204 may be of any other shape, such as square, triangular, rectangular, oval, elliptical, polygonal, or the like based on application attributes.

The test stack 112 further includes a sensor layer 206 spaced apart from the entrance layer 202. The sensor layer 206 defines a thickness T2 along the z-axis. In some cases, the thickness T2 of the sensor layer 206 is about 3 mil. In some embodiments, the thickness T2 of the sensor layer 206 is from about 10% to about 50% of the thickness T1 of the entrance layer 202. In some embodiments, each of the sensor layer 206 and the entrance layer 202 is impermeable to the steam sterilant. Therefore, each of the entrance layer 202 and the sensor layer 206 may not allow a fluid (e.g., steam) to pass therethrough.

The test stack 112 further includes an intermediate layer 208 disposed between the entrance layer 202 and the sensor layer 206. The intermediate layer 208 defines a thickness T3 along the z-axis. In some cases, the thickness T3 of the intermediate layer 208 is about 3 mil. In some embodiments, the thickness T3 of the intermediate layer 208 is from about 10% to about 50% of the thickness T1 of the entrance layer 202.

In the illustrated embodiment of FIG. 3, the intermediate layer 208 is spaced apart from the entrance layer 202 and disposed adjacent to the sensor layer 206. In other embodiments, the intermediate layer 208 may be disposed adjacent to the entrance layer 202, such that the intermediate layer 208 at least partially contacts the entrance layer 202. In some embodiments, the intermediate layer 208 is permeable or impermeable to the steam sterilant. In the illustrated embodiment of FIG. 3, the intermediate layer 208 includes a permeable material. Therefore, the intermediate layer 208 may allow a fluid (e.g., steam) to pass therethrough. In some embodiments, the permeable material includes nylon, clay, polyvinylidene difluoride (PVDF), soil loaded membranes, polypropylene blown microfiber (BMF), glass fiber, paper, clay loaded non-woven material, fine sand, or combinations thereof.

The test stack 112 further includes a first adhesive layer 210 disposed between the entrance layer 202 and the intermediate layer 208. The first adhesive layer 210 bonds the intermediate layer 208 to the entrance layer 202. The entrance hole 204 further extends through the first adhesive layer 210. In an example, the first adhesive layer 210 may include a very high bonding adhesive, such as a pressure sensitive adhesive, for example, but not limited to, silicone polyurea (SPU), acrylic, silicone, or rubber-based adhesive. In another example, the very high bonding adhesive may include structural adhesives, such as acrylic, cyanoacrylate, epoxy, polyurethane, or a mixture thereof. The first adhesive layer 210 defines a thickness T4 along the z-axis. In some cases, the thickness T4 of the first adhesive layer 210 is about 2 mil. In some embodiments, the thickness T4 of the first adhesive layer 210 is less than the thickness T3 of the intermediate layer 208.

The test stack 112 further includes a support layer 216 disposed adjacent to the sensor layer 206 opposite to the intermediate layer 208. The support layer 216 at least partially forms an external surface S2 of the test stack 112. The external surface S2 is disposed opposite to the external surface S1 formed by the entrance layer 202.

In some embodiments, the support layer 216 includes PET. In some other embodiments, the support layer 216 may be made of a metallic layer such as aluminum foil, a polymeric layer such as polyurethane or a polyester layer, without any limitations. The support layer 216 defines a thickness T5 along the z-axis. In some cases, the thickness T5 of the support layer 216 is about 10 mil. In some embodiments, the thickness T5 of the support layer 216 may be substantially equal to the thickness T1 of the entrance layer 202. In some embodiments, the support layer 216 is impermeable to the steam sterilant.

In some embodiments, the entrance layer 202, the intermediate layer 208, the sensor layer 206, and the support layer 216 at least together form a laminated construction. The test stack 112 further includes a second adhesive layer 218 disposed between the sensor layer 206 and the support layer 216. The second adhesive layer 218 bonds the support layer 216 to the sensor layer 206. The second adhesive layer 218 defines a thickness T6 along the z-axis. In some embodiments, the second adhesive layer 218 may have a thickness of about 2 mil. In some embodiments, the second adhesive layer 218 may include a very high bonding adhesive. In an example, the thickness T4 of the first adhesive layer 210 and the thickness T6 of the second adhesive layer 218 may be substantially equal to each other. In some embodiments, one or more layers of the test stack 112 may be transparent.

In some cases, various layers of the test stack 112 may be substantially co-extensive in length (i.e., along the y-axis) and width (i.e., along the x-axis) with each other. In some other cases, various layers of the test stack 112 may not be substantially co-extensive in length and width with each other. In the illustrated embodiment of FIG. 3, each of the entrance layer 202, the first adhesive layer 210, and the intermediate layer 208 have substantially equal length for illustrative purposes only.

It should be noted that edges of all the layers of the test stack 112 are sealed against each other to inhibit any fluidic connection between an internal volume of the test stack 112 and the chamber 104 via the edges of various layers.

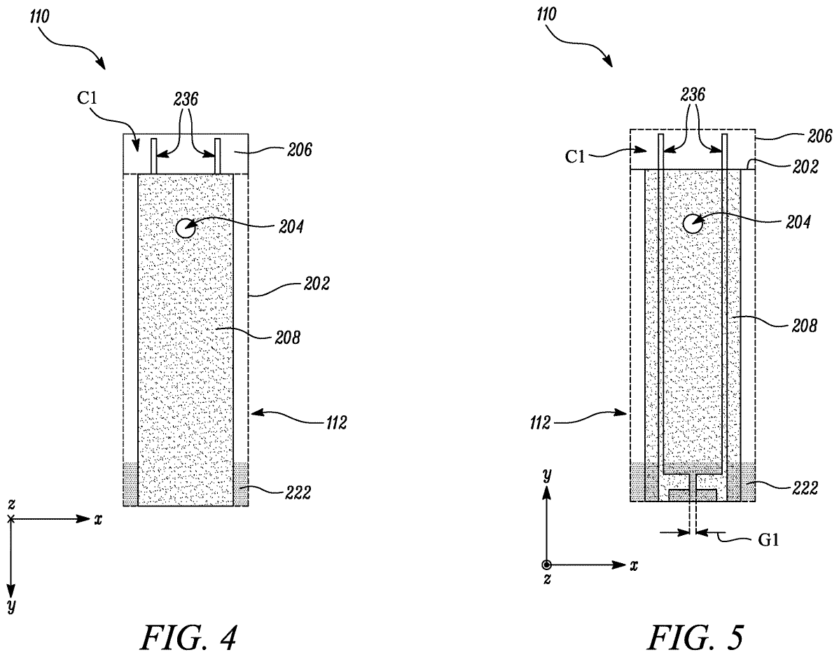

FIG. 4 illustrates a top view of the test device 110 including the test stack 112, according to an embodiment of the present disclosure. The entrance layer 202 is shown as transparent in FIG. 4 for illustrative purposes. Furter, the first adhesive layer 210 is not shown in FIG. 4 for illustrative purposes. In the illustrated embodiment of FIG. 4, the entrance layer 202 and the intermediate layer 208 have substantially equal length (i.e., along the y-axis). However, the entrance layer 202 and the intermediate layer 208 have unequal width (i.e., along the x-axis). In other embodiments, the entrance layer 202 and the intermediate layer 208 may be substantially co-extensive in length and width with each other.

FIG. 5 illustrates a bottom view of the test device 110, with some layers not shown, according to an embodiment of the present disclosure. Particularly, the support layer 216 and the second adhesive layer 218 are not shown in FIG. 5 for illustrative purposes. Further, the sensor layer 206 is shown as transparent in FIG. 5 for illustrative purposes.

Referring to FIGS. 4 and 5, the test stack 112 further includes at least one sensor coating 222 disposed on at least one portion of the sensor layer 206. The at least one sensor coating 222 includes an electrically active polymer. The at least one sensor coating 222 is spaced apart from the entrance hole 204 at least along the major plane A1 (shown in FIG. 2) of the test stack 112. Therefore, the at least one sensor coating 222 is spaced apart from the entrance hole 204 at least along the x-y plane of the test device 110.

The intermediate layer 208 fluidically connects the at least one sensor coating 222 and the entrance hole 204. Therefore, the intermediate layer 208 is configured to allow a flow of the steam sterilant received from the entrance hole 204 to the at least one sensor coating 222. Moreover, the intermediate layer 208 is configured to allow a flow of non-condensable gas (e.g., air) from the entrance hole 204 to the at least one sensor coating 222. As, the intermediate layer 208 includes the permeable material, the permeability of the permeable material is configured to allow the flow of the steam sterilant through the intermediate layer 208 in order to fluidically connect the entrance hole 204 with the at least one sensor coating 222.

The permeability of the permeable material of the intermediate layer 208 may offer a considerable resistance to flow of the steam sterilant through the intermediate layer 208. In some cases, the permeable material may provide the resistance that corresponds to a resistance provided by different routes and passages that the steam sterilant may have to follow to reach the hollow spaces and interior pockets of the medical equipment in a real-time sterilization process. The resistance provided by the permeable material may therefore represent the resistance of various flow channels leading to hidden spaces of tubes, catheters, syringe needles, and the like. The resistance provided by the permeable material of the intermediate layer 208 to the flow of the steam sterilant may depend on various properties of the permeable material.

In some embodiments, the electrically active polymer of the at least one sensor coating 222 includes polyaniline (PANI), trans polyacetylene, poly (p-phenylene), poly (3-vinylperlene), polypyrrole, poly (2,5-bis (3-tetradecylthiophene-2-yl) thieno [3,2-b] thiophene), poly (2-(3-thienyyloxy) ethanesulfonate), polythiophene, or combinations thereof.

In some embodiments, PANI may be in one of three oxidation states, i.e., leucoemeraldine, emeraldine (in a salt or base form), and per (nigraniline). The emeraldine may be less conductive in the base form and more conductive in the salt form. Further, the emeraldine salt may be converted into the leucoemeraldine salt or per (nigraniline) via a redox reaction to make the leucoemeraldine salt less conductive.

In some embodiments, the at least one sensor coating 222 further includes tin. In some cases, the at least one sensor coating 222 may include tin nanoparticles. In some other cases, the at least one sensor coating 222 may include PANI with blended nanoparticles of aluminum, transition metals, post transition metals, or combinations thereof.

With reference to FIGS. 1 to 5, the sensor layer 206 includes at least one pair of electrodes 236 disposed on the sensor layer 206. Further, the at least one sensor coating 222 is electrically coupled to the at least one pair of electrodes 236. In the illustrated embodiment, one pair of electrodes 236 is shown, however, the test device 110 may include any number of pairs of electrodes 236 as per application requirements. Each of the at least one pair of electrodes 236 may include a conductive material. In some embodiments, each of the at least one pair of electrodes 236 includes at least one of silver, carbon and aluminum.

In some embodiments, at least a portion of each of the at least one pair of electrodes 236 is disposed between the at least one sensor coating 222 and the sensor layer 206, such that at least one gap G1 is defined between the at least one pair of electrodes 236. The at least one gap G1 is covered by the at least one sensor coating 222.

In some embodiments, the entrance layer 202 and the intermediate layer 208 at least partially define a cutout C1 disposed at the perimeter P of the test stack 112. Each of the at least one pair of electrodes 236 at least partially extends into the cutout C1.

In some embodiments, the sterilization monitoring system 106 may further include a holder (not shown) configured to at least partially and removably receive the test device 110 therein. The holder may further be configured to removably secure or hold the test device 110. For conducting a sterilization monitoring cycle, the holder and the test device 110 at least partially received within the holder may be

The sterilization monitoring system 106 including the test device 110 may also be used in other sterilization modalities, such as vaporized hydrogen peroxide sterilization. Moreover, the sterilization monitoring system 106 may be used in different types of steam sterilizers that are already manufactured and are being currently used in the medical industry.

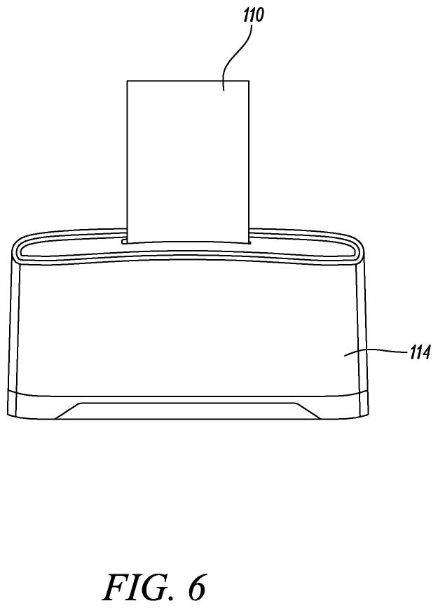

The sterilization monitoring system 106 further includes a reader 114 (shown in FIG. 1) configured to at least partially receive the test device 110 therein for measuring an electrical impedance I1 across the at least one pair of electrodes 236. FIG. 6 schematically shows the reader 114, according to an embodiment of the present disclosure. Specifically, in FIG. 6, the test device 110 is received in the reader 114. A value of the electrical impedance I1 may be stored in a memory 116 of the reader 114.

Referring to FIGS. 1 to 6, the cutout C1 is configured to at least partially receive one or more terminals (not shown) of the reader 114 therein for measuring the electrical impedance I1 across the at least one pair of electrodes 236. Further, the at least one sensor coating 222 is configured to change the electrical impedance I1 across the at least one pair of electrodes 236 upon contact of the steam sterilant with the at least one sensor coating 222. In some embodiments, upon contact with the steam sterilant, the at least one sensor coating 222 is further configured to change the electrical impedance I1 across the at least one pair of electrodes 236 beyond a predetermined threshold impedance 12 (may be stored in the memory 116).

Further, it should be noted that the electrically active polymer in the at least one sensor coating 222 switches between one impedance state and another impedance state based on an interaction with the steam sterilant. In some embodiments, as the at least one pair of electrodes 236 may be coated with or formed from the conductive material, such as silver, carbon or aluminum, the conductive material may directly react with the at least one sensor coating 222 and convert emeraldine salt into leucoemeraldine salt to make the leucoemeraldine salt less conductive. The at least one sensor coating 222 may therefore change from one impedance state to another impedance state based on the redox reaction of the electrically active polymer with the conductive material of the at least one pair of electrodes 236 at the environmental condition of the chamber 104.

Moreover, in some embodiments, upon the appropriate exposure of the steam sterilant to the at least one sensor coating 222, the at least one pair of electrodes 236 may switch from being electrically shorted, i.e., a small impedance between the at least one pair of electrodes 236 to being in an electrically open condition, i.e., a large impedance between the at least one pair of electrodes 236.

While monitoring sterilization, the reader 114 provides a pass result upon determining that the electrical impedance I1 across the at least one pair of electrodes 236 is beyond the predetermined threshold impedance I2. Further, the reader 114 provides a fail result upon determining that the electrical impedance I1 across the at least one pair of electrodes 236 is below the predetermined threshold impedance I2. Therefore, the reader 114 may provide an accurate pass or fail result of a steam sterilization process based on a comparison between the predetermined threshold impedance 12 and the electrical impedance I1 across the at least one pair of electrodes 236.

In cases where the electrical impedance I1 across the at least one pair of electrodes 236 is beyond the predetermined threshold impedance 12, an operator may also determine a quantitative relevancy of the pass result based on a magnitude of a difference between the electrical impedance I1 and the predetermined threshold impedance I2. In cases where the electrical impedance I1 across the at least one pair of electrodes 236 is not beyond the predetermined threshold impedance 12, the operator may also determine a quantitative relevancy of the fail result based on the magnitude of the difference between the electrical impedance I1 and the predetermined threshold impedance I2.

Further, the test device 110 is a built-in and a stand-alone unit which can be used with any sterilizer. In contrast to a conventional technique of monitoring sterilization by using test packs and/or indicator sheets, and then manually interpreting the change in color of the indicator sheets, the sterilization monitoring system 106 including the test device 110 may require minimal human interpretation to determine the pass/fail result of the sterilization process. Therefore, the test device 110 and the sterilization monitoring system 106 of the present disclosure may provide an accurate classification of test results that could have been otherwise erroneous due to possible human intervention errors. Moreover, as the test device 110 is being used here for monitoring steam quality of the steam sterilant by measuring the electrical impedance I1 across the at least one pair of electrodes 236, the test device 110 of the present disclosure may be called as an electronic testing unit or an electronic test card. In some cases, the sterilization monitoring system 106 including the test device 110 and the reader 114 may also provide a digital pass/fail result of the steam quality of the steam sterilant.

In contrast to conventional techniques for monitoring sterilization, the sterilization monitoring system 106 of the present disclosure may eliminate a need for scanning of images of the test packs (indicator sheets), photocopying the test results, and manually recording the test results. Moreover, the sterilization monitoring system 106 including the test device 110 may eliminate the need to maintain a record/logbook of Bowie-Dick test results of one or more sterilizers. This may also reduce a possibility of misplacing the various test results of the steam quality of the steam sterilant. Therefore, an overall throughput of the sterilizer 102 may be increased due to minimal manual recording and/or manual maintenance of the test results. The sterilization monitoring system 106 may allow a faster and an easier testing process for the operator to accurately monitor the steam quality of the steam sterilant and/or validate proper air removal in the chamber 104 of the sterilizer 102. Consequently, the disclosed sterilization monitoring system 106 may increase an efficiency of the sterilizer 102 and decrease a complexity of the process to monitor the steam quality of the steam sterilant. Moreover, the sterilization monitoring system 106 may also save a large amount of paper that was otherwise wasted in the conventional techniques for monitoring sterilization.

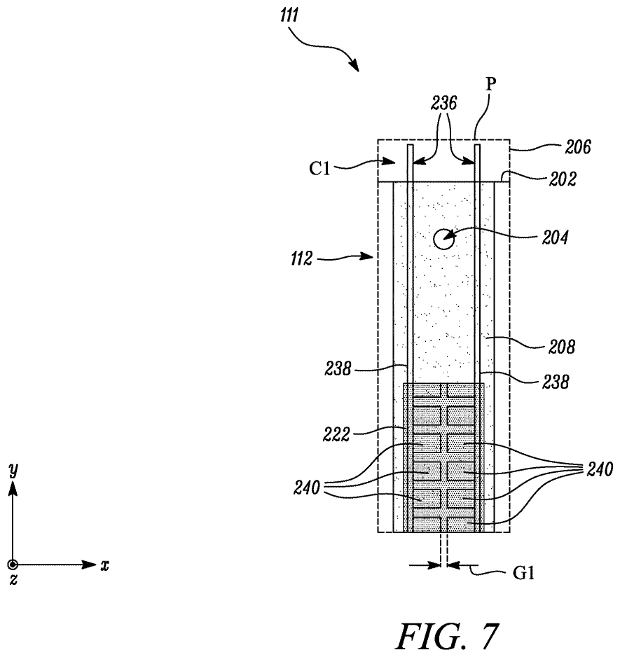

FIG. 7 illustrates a bottom view of a test device 111, according to another embodiment of the present disclosure. The test device 111 is substantially similar to the test device 110 illustrated in FIGS. 4 and 5, with common components being referred to by the same reference numerals. In the illustrated embodiment of FIG. 7, the support layer 216 and the second adhesive layer 218 are not shown for illustrative purposes. Further, the sensor layer 206 is shown as transparent in FIG. 7 for illustrative purposes. Further, a functional advantage of the test device 111 is substantially same as that of the test device 110.

In the test device 111, each electrode 236 of the at least one pair of electrodes 236 includes an elongate portion 238 extending from the at least one sensor coating 222 towards the perimeter P of the test stack 112 and a plurality of projections 240 extending from and inclined to the elongate portion 238. In the illustrated embodiment of FIG. 7, the plurality of projections 240 are disposed in the major plane A1 of the test stack 112 and extend perpendicularly from the elongate portion 238 of each of the at least one pair of electrodes 236. Each projection 240 has a substantially rectangular shape in FIG. 7. However, each projection 240 may have any suitable alternative shape, for example, triangular, elliptical, polygonal, oval, circular, and the like.

The plurality of projections 240 of one of the at least one pair of electrodes 236 and the projections 240 of the other of the at least one pair of electrodes 236 extend towards each other and define a plurality of gaps G1 therebetween. In other words, the plurality of projections 240 extending from one of the elongate portions 238 of the at least one pair of electrodes 236 and the plurality of projections 240 extending from the elongate portions 238 of the other of the at least one pair of electrodes 236 extend towards each other and define the plurality of gaps G1 therebetween. The plurality of projections 240 of each electrode 236 of the at least one pair of electrodes 236 form a ladder type configuration.

Moreover, the plurality of gaps G1 are shown equal to each other for illustrative purposes. However, in some cases, the plurality of gaps G1 may also be different from each other. Further, each gap G1 from the plurality of gaps G1 is defined between a corresponding projection 240 from the plurality of projections 240 of the one of the at least one pair of electrodes 236 and a corresponding projection 240 from the plurality of projections 240 of the other of the at least one pair of electrodes 236.

In the illustrated embodiment of FIG. 7, the at least one sensor coating 222 is a single sensor coating 222. Further, the at least one pair of electrodes 236 is a single pair of electrodes 236. Therefore, in the test device 111, the plurality of gaps G1 are covered by the single sensor coating 222.

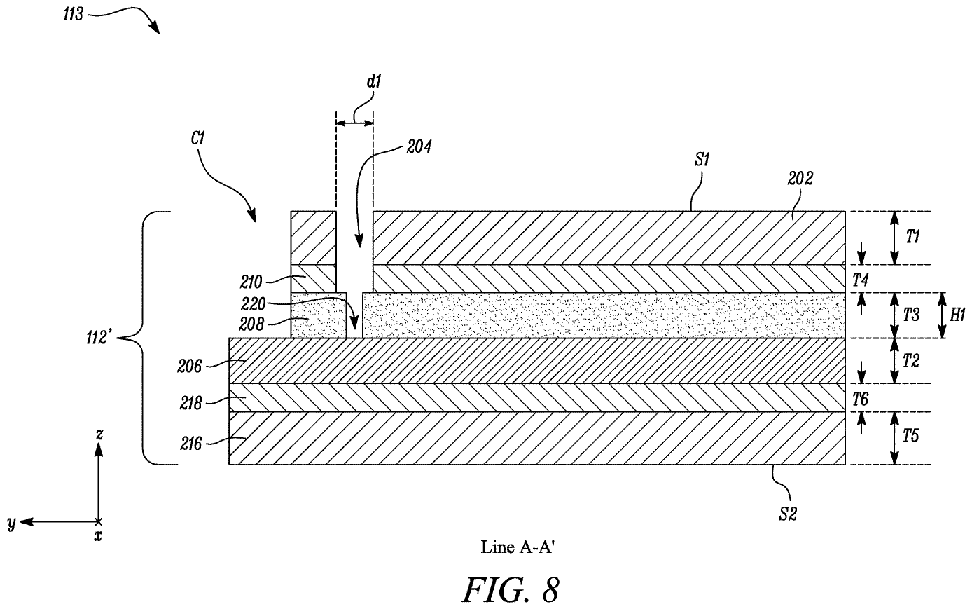

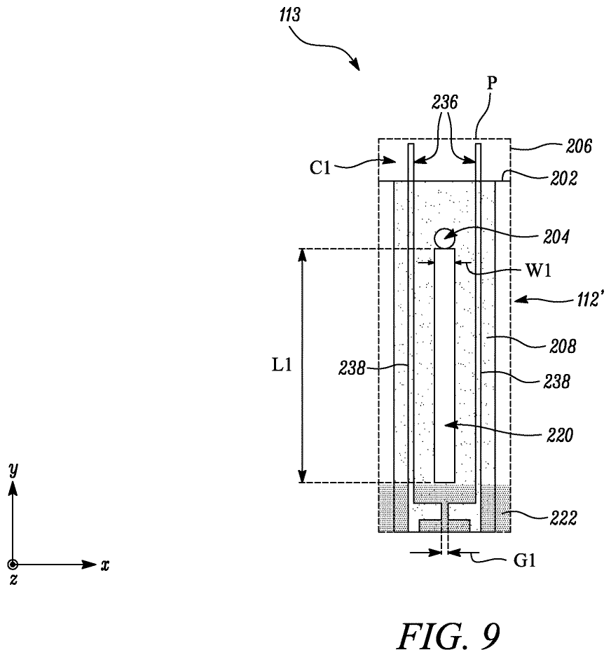

FIG. 8 is a sectional side view of a test device 113, according to another embodiment of the present disclosure. The test device 113 is substantially similar to the test device 110 illustrated in FIG. 3, with common components being referred to by the same reference numerals. Further, the test device 113 includes a test stack 112′substantially similar to the test stack 112 of the test device 110 illustrated in FIG. 3. The sectional side view of the test device 113 is taken along the line A-A′ shown in FIG. 2. FIG. 9 illustrates a bottom view of the test device 113, with some layers not shown. Particularly, the support layer 216 and the second adhesive layer 218 are not shown in FIG. 9 for illustrative purposes. Further, the sensor layer 206 is shown as transparent in FIG. 9 for illustrative purposes. Moreover, a functional advantage of the test device 113 is substantially same as that of the test device 110.

Referring to FIGS. 8 and 9, in the test stack 112′of the test device 113, the intermediate layer 208 of the test device 113 includes at least one internal channel 220 defining a channel length L1 (shown in FIG. 9) along the major plane A1 and a channel depth H1 normal to the major plane A1 (shown in FIG. 2). In some embodiments, the at least one internal channel 220 may be interchangeably referred to as “at least one intermediate path 220”. In some embodiments, the channel length L1 may be interchangeably referred to as “path length L1”. In some embodiments, the channel depth H1 may be interchangeably referred to as “path depth H1”. The at least one internal channel 220 is spaced apart from the perimeter P of the test stack 112′. The at least one internal channel 220 extends through the intermediate layer 208 along the channel depth H1. Further, the at least one internal channel 220 extends from the entrance hole 204 to the at least one sensor coating 222 at least along the channel length L1, such that the at least one internal channel 220 fluidically connects the entrance hole 204 with the at least one sensor coating 222. Therefore, the at least one internal channel 220 is configured to allow a flow of the steam sterilant from the entrance hole 204 to the at least one sensor coating 222. Moreover, the at least one internal channel 220 is also configured to allow the flow of non-condensable gas (e.g., air) from the entrance hole 204 to the at least one sensor coating 222.

The at least one internal channel 220 defines a width W1 extending perpendicularly to the channel depth H1. In some embodiments, the width W1 of the at least one internal channel 220 is less than or equal to the diameter dl of the entrance hole 204. In the illustrated embodiment of FIG. 9, the at least one internal channel 220 is linear. In some other embodiments, the at least one internal channel 220 may be at least partially non-linear along the channel length L1. Moreover, the shape and the dimensions of the at least one internal channel 220 may vary based on different application attributes.

The at least one internal channel 220 may offer a considerable resistance to the flow of the steam sterilant through the at least one internal channel 220. Particularly, the resistance provided by the at least one internal channel 220 may correspond to the resistance provided by the different routes and the passages that the steam sterilant may have to follow to reach the hollow spaces and interior pockets of the medical equipment in a real-time sterilization process. The resistance provided by the at least one internal channel 220 to the flow of the steam sterilant may depend on a shape and dimensions of the at least one internal channel 220.

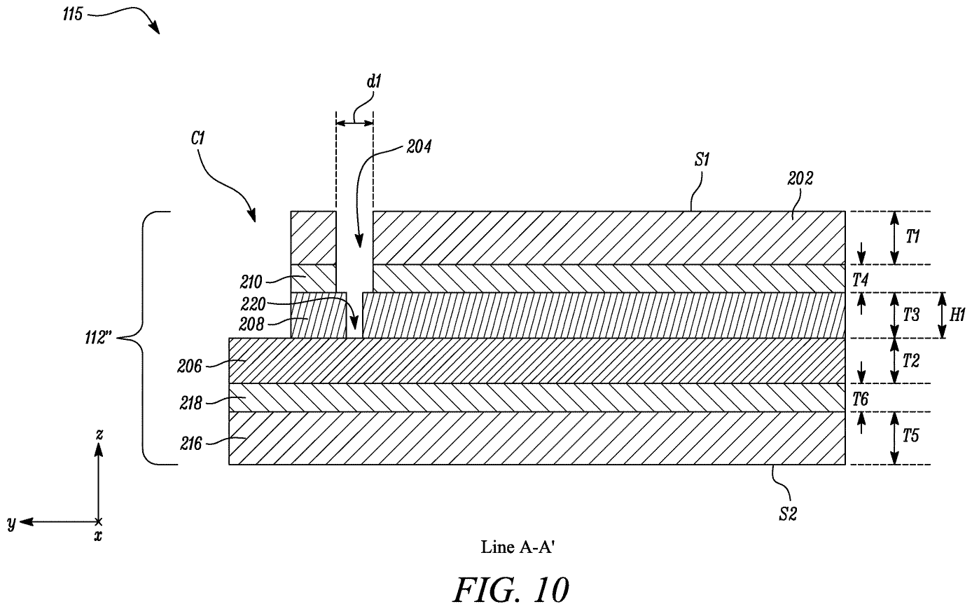

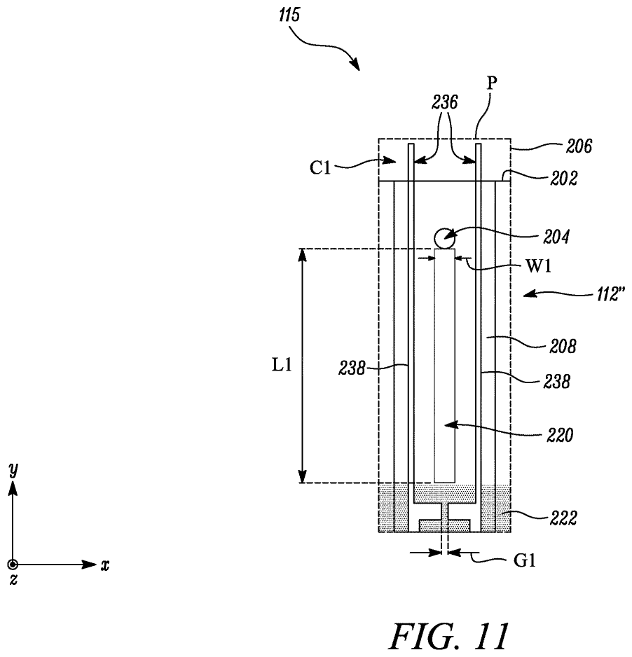

FIG. 10 is a sectional side view of a test device 115, according to another embodiment of the present disclosure. The test device 115 is substantially similar to the test device 113 illustrated in FIG. 8, with common components being referred to by the same reference numerals. Further, the test device 115 includes a test stack 112″ substantially similar to the test stack 112′of the test device 113 illustrated in FIG. 8. The sectional side view of the test device 115 is taken along the line A-A′ shown in FIG. 2. FIG. 11 illustrates a bottom view of the test device 115, with some layers not shown. Particularly, the support layer 216 and the second adhesive layer 218 are not shown in FIG. 11 for illustrative purposes. Further, the sensor layer 206 is shown as transparent in FIG. 11 for illustrative purposes. Moreover, a functional advantage of the test device 115 is substantially same as that of the test device 113.

Referring to FIGS. 10 and 11, in the test device 115, the intermediate layer 208 includes an impermeable material that may not allow a fluid (e.g., steam) to pass therethrough. Therefore, steam flowing through the entrance hole 204 has to flow through the at least one internal channel 220 to reach the at least one sensor coating 222. In some embodiments, the intermediate layer 208 of the test stack 112″ includes PET.

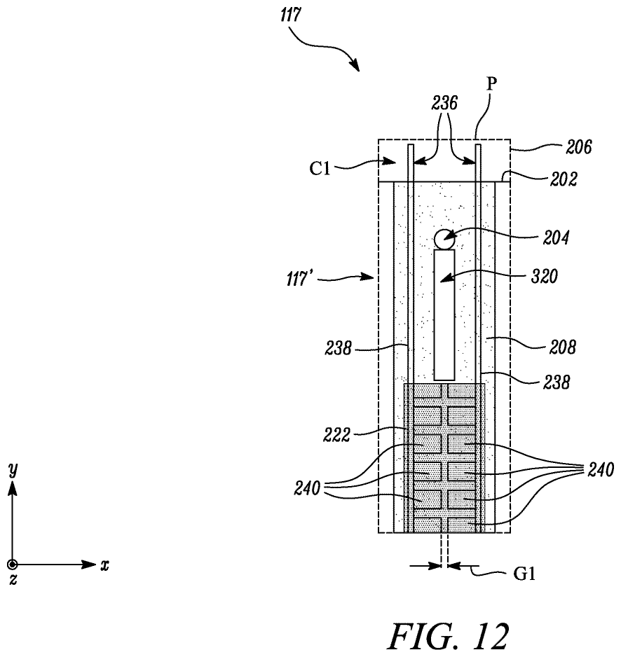

FIG. 12 illustrates a bottom view of a test device 117, with some layers not shown, according to another embodiment of the present disclosure. The test device 117 is substantially similar to the test device 111 illustrated in FIG. 7, with common components being referred to by the same reference numerals. Further, the test device 117 includes a test stack 117′substantially similar to the test stack 112 of the test device 110 illustrated in FIG. 3. In the illustrated embodiment of FIG. 12, the support layer 216 and the second adhesive layer 218 are not shown for illustrative purposes. Further, the sensor layer 206 is shown as transparent in FIG. 12 for illustrative purposes. Moreover, a functional advantage of the test device 117 is substantially same as that of the test device 111.

In comparison to the test device 111 of FIG. 7, the test device 117 further includes at least one internal channel 320 extending from the entrance hole 204 to the at least one sensor coating 222. In some embodiments, geometrical characteristics of the at least one internal channel 320 is substantially same as that of the at least one internal channel 220 of the test device 113 of FIGS. 8 and 9. A length of the at least one internal channel 320 may be different from the channel length L1 (shown in FIG. 11) of the at least one internal channel 220. In the illustrated embodiment of FIG. 12, the intermediate layer 208 is permeable. In other embodiments, the intermediate layer 208 may be impermeable.

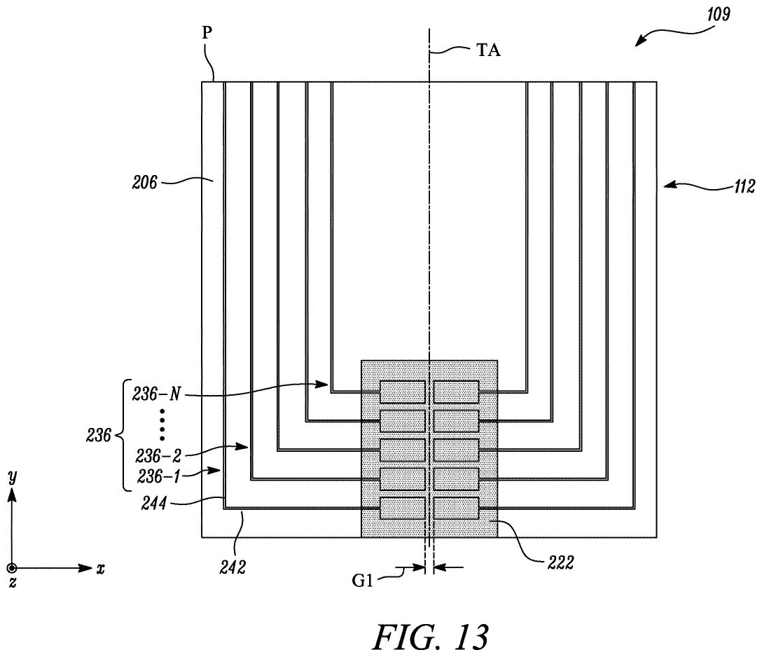

FIG. 13 is a bottom view of a test device 109, with some layers not shown, according to another embodiment of the present disclosure. The test device 109 is substantially similar to the test device 110 illustrated in FIGS. 3 to 5, with common components being referred to by the same reference numerals. In FIG. 13, only the at least one pair of electrodes 236, the sensor layer 206, and the at least one sensor coating 222 are shown for illustrative purposes. Moreover, a functional advantage of the test device 109 is substantially same as that of the test device 110 of FIGS. 3 to 5.

In the test device 109 of FIG. 13, the at least one pair of electrodes 236 includes a plurality of pairs of electrodes 236-1, 236-2 . . . 236-N (collectively referred to as “pairs of electrodes 236”) defining a plurality of gaps G1. In some embodiments, the plurality of pairs of electrodes 236-1, 236-2 . . . 236-N are spaced apart from each other in the major plane A1. In some embodiments, the plurality of pairs of electrodes 236-1, 236-2 . . . 236-N are disposed adjacent to each other along an elongate axis TA.

Further, each of the plurality of gaps G1 is defined between one electrode 236 of a corresponding pair of electrodes 236 from the plurality of pairs of electrodes 236-1, 236-2 . . . 236-N and the other electrode 236 of the corresponding pair of electrodes 236 from the plurality of pairs of electrodes 236-1, 236-2 . . . 236-N.

With continued reference to FIG. 13, each electrode 236 of the plurality of pairs of electrodes 236-1, 236-2 . . . 236-N includes a first portion 242 extending from the at least one sensor coating 222 and an orthogonal second portion 244 extending from the first portion 242 towards the perimeter P. Each of the plurality of gaps G1 is defined between the first portions 242 of the corresponding pair of electrodes 236. In the illustrated embodiment of FIG. 13, the at least one sensor coating 222 is a single sensor coating 222. The plurality of gaps G1 are covered by the single sensor coating 222. It should be noted that only five pairs of electrodes 236 are illustrated in FIG. 13. However, in some other embodiments, the at least one pair of electrodes 236 may include any number of pair of electrodes 236.

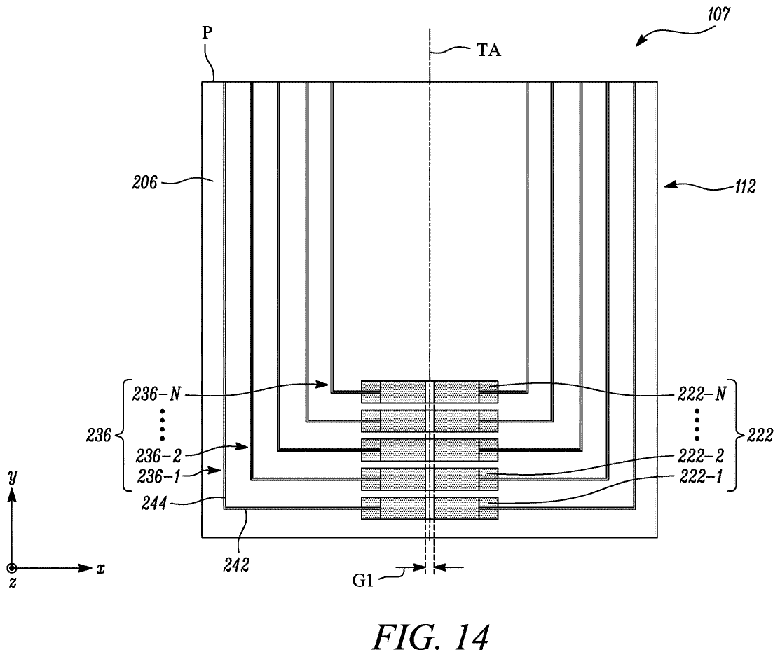

FIG. 14 is a bottom view of a test device 107, with some layers not shown, according to another embodiment of the present disclosure. The test device 107 is substantially similar to the test device 109 illustrated in FIG. 13, with common components being referred to by the same reference numerals. In FIG. 14, only the at least one pair of electrodes 236, the sensor layer 206, and the at least one sensor coating 222 are shown for illustrative purposes. Moreover, a functional advantage of the test device 107 is substantially same as that of the test device 109 of FIG. 13.

In the test device 107, the at least one sensor coating 222 includes a plurality of sensor coatings 222-1, 222-2 . . . 222-N (collectively referred to as “sensor coatings 222”) corresponding to the plurality of gaps G1 and spaced apart from each other. Each of the plurality of gaps G1 is covered by a corresponding sensor coating 222 from the plurality of sensor coatings 222-1, 222-2 . . . 222-N. Further, in the illustrated embodiment of FIG. 14, the plurality of pairs of electrodes 236-1, 236-2 . . . 236-N are disposed adjacent to each other along the elongate axis TA, such that the plurality of gaps G1 and the plurality of sensor coatings 222-1, 222-2 . . . 222-N are arranged along the elongate axis TA.

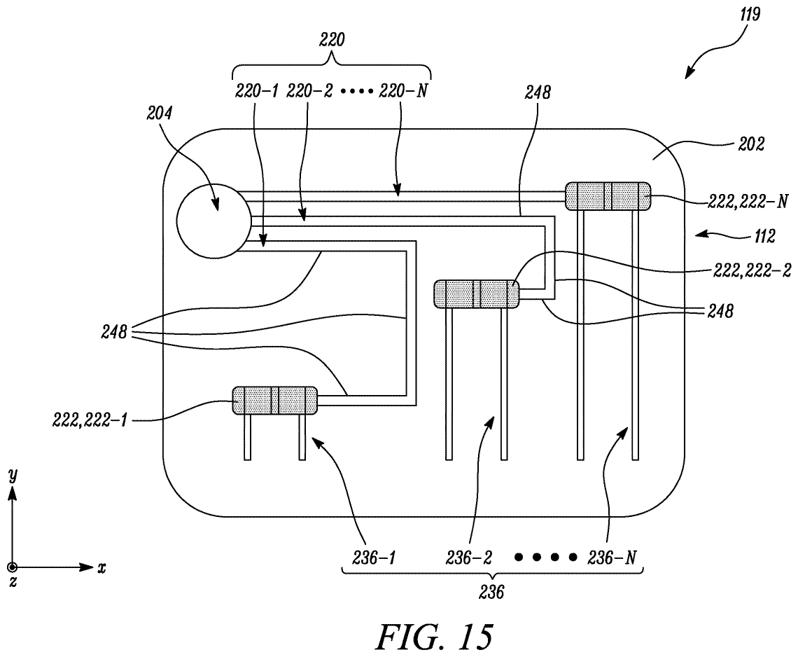

FIG. 15 illustrates a bottom view of a test device 119, with some layers not shown, according to another embodiment of the present disclosure. The test device 119 is functionally equivalent to the test device 113 illustrated in FIGS. 8 and 9, with common components being referred to by the same reference numerals. Further, functional advantage of the test device 119 is substantially same as that of the test device 113 of FIGS. 8 and 9. In the illustrated embodiment of FIG. 15, the support layer 216 and the second adhesive layer 218 are not shown for illustrative purposes. Further, the sensor layer 206, the intermediate layer 208, and the first adhesive layer 210 are shown as transparent in FIG. 15 for illustrative purposes.

In the test device 119, the at least one internal channel 220 includes a plurality of linear portions 248 connected to each other. Further, adjacent linear portions 248 from the plurality of linear portions 248 are inclined to each other. In some embodiments, adjacent linear portions 248 may be perpendicular to each other. In some cases, the plurality of linear portions 248 may be of different lengths relative to each other.

Further, in the test device 119, the at least one pair of electrodes 236 includes the plurality of pairs of electrodes 236-1, 236-2 . . . 236-N (also illustrated in FIG. 14) spaced apart from each other in the major plane A1 (shown in FIG. 2). The at least one sensor coating 222 includes the plurality of sensor coatings 222-1, 222-2 . . . 222-N (also illustrated in FIG. 14) corresponding to the plurality of pairs of electrodes 236-1, 236-2 . . . 236-N and spaced apart from each other. Each of the plurality of sensor coatings 222-1, 222-2 . . . 222-N is electrically coupled to a corresponding pair of electrodes 236 from the plurality of pairs of electrodes 236-1, 236-2 . . . 236-N.

Moreover, in the test device 119, the at least one internal channel 220 includes a plurality of internal channels 220-1, 220-2 . . . 220-N (collectively referred to as “internal channels 220”) corresponding to the plurality of sensor coatings 222-1, 222-2 . . . 222-N and spaced apart from each other. Each internal channel 220 from the plurality of internal channels 220-1, 220-2 . . . 220-N fluidically connects the entrance hole 204 with a corresponding sensor coating 222 from the plurality of sensor coatings 222-1, 222-2 . . . 222-N. In the illustrated embodiment of FIG. 15, the at least one internal channel 220 includes three internal channels 220 in total. In other embodiments, the at least one internal channel 220 may include any number of internal channels 220. The shape and the dimensions of the plurality of internal channels 220-1, 220-2 . . . 220-N may vary based on different application attributes.

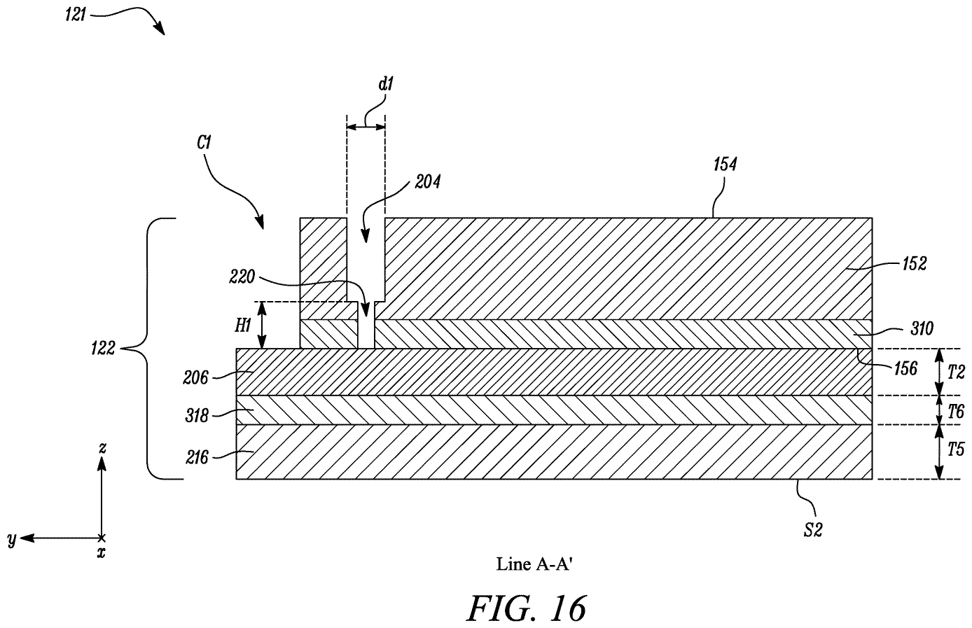

FIG. 16 is a sectional view of a test device 121, according to another embodiment of the present disclosure. The test device 121 is substantially similar to the test device 113 illustrated in FIGS. 8 and 9, with common components being referred to by the same reference numerals. Further, the test device 121 includes a test stack 122 substantially similar to the test stack 112′of the test device 113 illustrated in FIG. 8, with common components being referred to by the same reference numerals.

However, in the test device 121, the test stack 122 includes a top layer 152 (instead of a combination of the entrance layer 202, the first adhesive layer 210, and the intermediate layer 208 in the test stack 112 of FIG. 3) including a first major surface 154 proximal to the chamber 104 and a second major surface 156 opposite to the first major surface 154. The top layer 152 further includes the entrance hole 204 extending from the first major surface 154 at least partially through the top layer 152 and disposed in fluidic connection with the chamber 104. The top layer 152 incorporates the at least one intermediate path 220 at least partially aligned with and disposed in fluidic connection with the entrance hole 204. The at least one intermediate path 220 extends from the second major surface 156 at least partially through the top layer 152 along the path depth H1. The at least one intermediate path 220 is spaced apart from the perimeter P of the test stack 122.

Further, the sensor layer 206 is disposed adjacent to the second major surface 156 of the top layer 152. The entrance hole 204 is disposed in fluidic connection with the at least one sensor coating 222 (shown in FIGS. 4 and 5). Furthermore, the at least one intermediate path 220 of the top layer 152 extends from the entrance hole 204 to the at least one sensor coating 222 at least along the path length L1 (shown in FIG. 9), such that the at least one intermediate path 220 fluidically connects the entrance hole 204 with the at least one sensor coating 222.

The top layer 152 is configured to allow the flow of the steam sterilant received from the entrance hole 204 to the at least one sensor coating 222. Therefore, in presence of the steam sterilant or in absence of air of any non-condensable gas, the at least one sensor coating 222 is configured to change the electrical impedance I1 (shown in FIG. 1) across the at least one pair of electrodes 236 upon contact of the steam sterilant with the at least one sensor coating 222. In some embodiments, at least some portion of the top layer 152 may be permeable to the steam sterilant to fluidically connect the entrance hole 204 with the sensor layer 206.

In some embodiments, the test stack 122 further includes the support layer 216 disposed adjacent to the sensor layer 206 and opposite to the top layer 152. The support layer 216 at least partially forms the external surface S2 of the test stack 122. In some embodiments, the test stack 122 further includes an adhesive layer 318 disposed between the sensor layer 206 and the support layer 216. The adhesive layer 318 bonds the support layer 216 to the sensor layer 206. In some examples, the adhesive layer 318 is substantially similar to the second adhesive layer 218 (shown in FIG. 8). In some embodiments, the test stack 122 further includes an adhesive layer 310 disposed between the sensor layer 206 and the top layer 152. The adhesive layer 310 bonds the top layer 152 to the sensor layer 206. The adhesive layer 310 is substantially similar to the first adhesive layer 210 (shown in FIG. 8).

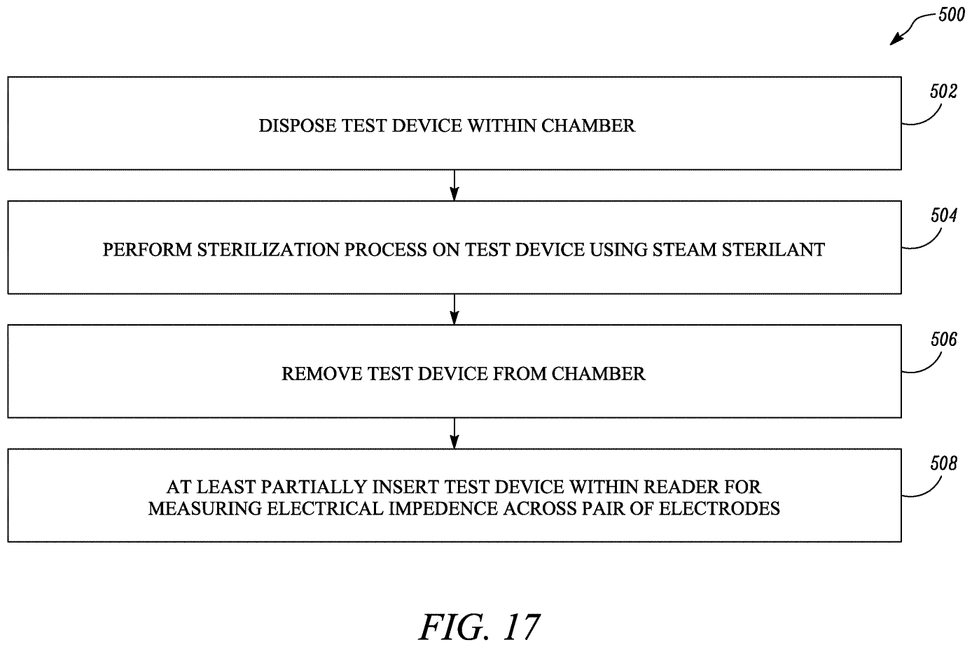

FIG. 17 illustrates a flowchart for a method 500 for monitoring sterilization in the chamber 104 (shown in FIG. 1) using the test device 110 (shown in FIG. 2). The method 500 may also be implemented by the test device 111 (shown in FIG. 7), the test device 113 (shown in FIG. 8), the test device 115 (shown in FIG. 10), the test device 117 (shown in FIG. 12), the test device 109 (shown in FIG. 13), the test device 107 (shown in FIG. 14), the test device 119 (shown in FIG. 15), and the test device 121 (shown in FIG. 16).

With reference to FIGS. 2 and 17, at step 502, the method 500 includes disposing the test device 110 within the chamber 104. At step 504, the method 500 includes performing the sterilization process on the test device 110 using the steam sterilant. At step 506, the method 500 includes removing the test device 110 from the chamber 104. At step 508, the method 500 includes at least partially inserting the test device 110 within the reader 114 for measuring the electrical impedance I1 across the pair of electrodes 236.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A test device for monitoring sterilization using a steam sterilant in a chamber, the test device comprising:
a test stack defining a major plane and a perimeter, the test stack comprising:
an entrance layer comprising an entrance hole extending through the entrance layer, wherein the entrance hole is in fluidic connection with the chamber;
a sensor layer spaced apart from the entrance layer, wherein the sensor layer comprises at least one pair of electrodes disposed on the sensor layer;
at least one sensor coating disposed on at least one portion of the sensor layer and comprising an electrically active polymer, wherein the at least one sensor coating is spaced apart from the entrance hole at least along the major plane of the test stack, wherein the at least one sensor coating is electrically coupled to the at least one pair of electrodes; and
an intermediate layer disposed between the entrance layer and the sensor layer, wherein the intermediate layer fluidically connects the entrance hole and the at least one sensor coating;
wherein the intermediate layer is configured to allow a flow of the steam sterilant received from the entrance hole to the at least one sensor coating, and wherein the at least one sensor coating is configured to change an electrical impedance across the at least one pair of electrodes upon contact of the steam sterilant with the at least one sensor coating.

2. The test device of claim 1, wherein the intermediate layer is spaced apart from the entrance layer and disposed adjacent to the sensor layer.

3. The test device of claim 1, wherein the entrance layer and the intermediate layer at least partially define a cutout disposed at the perimeter of the test stack, wherein each of the at least one pair of electrodes at least partially extends into the cutout, and wherein the cutout is configured to at least partially receive one or more terminals of a reader therein for measuring the electrical impedance across the at least one pair of electrodes.

4. The test device of claim 1, wherein at least a portion of each of the at least one pair of electrodes is disposed between the at least one sensor coating and the sensor layer, such that at least one gap is defined between the at least one pair of electrodes, and wherein the at least one gap is covered by the at least one sensor coating.

5. The test device of claim 1, wherein each electrode of the at least one pair of electrodes comprises an elongate portion extending from the at least one sensor coating towards the perimeter of the test stack and a plurality of projections extending from and inclined to the elongate portion, wherein the plurality of projections of one of the at least one pair of electrodes and the plurality of projections of the other of the at least one pair of electrodes extend towards each other and define a plurality of gaps therebetween, and wherein each gap from the plurality of gaps is defined between a corresponding projection from the plurality of projections of the one of the at least one pair of electrodes and a corresponding projection from the plurality of projections of the other of the at least one pair of electrodes.

6. The test device of claim 5, wherein the at least one sensor coating is a single sensor coating, wherein the at least one pair of electrodes is a single pair of electrodes, and wherein the plurality of gaps are covered by the single sensor coating.

7. The test device of claim 5, wherein the plurality of projections are disposed in the major plane of the test stack and extend perpendicularly from the elongate portion of each of the at least one pair of electrodes.

8. The test device of claim 1, wherein the at least one pair of electrodes comprises a plurality of pairs of electrodes defining a plurality of gaps, and wherein each of the plurality of gaps is defined between one electrode of a corresponding pair of electrodes from the plurality of pairs of electrodes and the other electrode of the corresponding pair of electrodes from the plurality of pairs of electrodes.

9. The test device of claim 8, wherein each electrode of the plurality of pairs of electrodes comprises a first portion extending from the at least one sensor coating and an orthogonal second portion extending from the first portion towards the perimeter, and wherein each of the plurality of gaps is defined between the first portions of the corresponding pair of electrodes.

10. The test device of claim 8, wherein the at least one sensor coating is a single sensor coating, and wherein the plurality of gaps are covered by the single sensor coating.

11. The test device of claim 8, wherein the at least one sensor coating comprises a plurality of sensor coatings corresponding to the plurality of gaps and spaced apart from each other, and wherein each of the plurality of gaps is covered by a corresponding sensor coating from the plurality of sensor coatings.

12. The test device of claim 11, wherein the plurality of pairs of electrodes are disposed adjacent to each other along an elongate axis, such that the plurality of gaps and the plurality of sensor coatings are arranged along the elongate axis.

13. The test device of claim 1, wherein the intermediate layer comprises a permeable material, and wherein the permeability of the permeable material of the intermediate layer is configured to allow the flow of the steam sterilant through the intermediate layer in order to fluidically connect the entrance hole with the at least one sensor coating.

14. (canceled)

15. The test device of claim 1, wherein the intermediate layer further comprises at least one internal channel defining a channel length along the major plane and a channel depth normal to the major plane, wherein the at least one internal channel is spaced apart from the perimeter of the test stack, wherein the at least one internal channel extends through the intermediate layer along the channel depth, wherein the at least one internal channel extends from the entrance hole to the at least one sensor coating at least along the channel length, such that the at least one internal channel fluidically connects the entrance hole with the at least one sensor coating.

16-18. (canceled)

19. The test device of claim 15, wherein the at least one pair of electrodes comprises a plurality of pairs of electrodes spaced apart from each other in the major plane, wherein the at least one sensor coating comprises a plurality of sensor coatings corresponding to the plurality of pairs of electrodes and spaced apart from each other, wherein each of the plurality of sensor coatings is electrically coupled to a corresponding pair of electrodes from the plurality of pairs of electrodes, wherein the at least one internal channel comprises a plurality of internal channels corresponding to the plurality of sensor coatings and spaced apart from each other, and wherein each internal channel from the plurality of internal channels fluidically connects the entrance hole with a corresponding sensor coating from the plurality of sensor coatings.

20-31. (canceled)

32. The test device of claim 1, wherein, upon contact with the steam sterilant, the at least one sensor coating is further configured to change the electrical impedance across the at least one pair of electrodes beyond a predetermined threshold impedance.

33. (canceled)

34. A sterilization monitoring system comprising;
the test device of claim 1; and
a reader configured to at least partially receive the test device therein for measuring the electrical impedance across the at least one pair of electrodes.

35. A sterilization system comprising;
the sterilization monitoring system of claim 34; and
a sterilizer comprising a chamber configured to receive the test device therein, wherein the sterilizer is configured to perform a sterilization process on the test device using a steam sterilant within the chamber.

36. A method for monitoring sterilization in a chamber using the test device of claim 1, the method comprising:
disposing the test device within the chamber;
performing a sterilization process on the test device using a steam sterilant;
removing the test device from the chamber; and
at least partially inserting the test device within a reader for measuring the electrical impedance across the at least one pair of electrodes.

37. A test device for monitoring sterilization using a steam sterilant in a chamber, the test device comprising:
a test stack defining a major plane and a perimeter, the test stack comprising:
a top layer comprising a first major surface proximal to the chamber, a second major surface opposite to the first major surface, and an entrance hole extending from the first major surface at least partially through the top layer and disposed in fluidic connection with the chamber, and incorporating at least one intermediate path at least partially aligned with and disposed in fluidic connection with the entrance hole, the at least one intermediate path defining a path length along the major plane and a path depth normal to the major plane, wherein the at least one intermediate path extends from the second major surface at least partially through the top layer along the path depth, and wherein the at least one intermediate path is spaced apart from the perimeter of the test stack;
a sensor layer disposed adjacent to the second major surface of the top layer, wherein the sensor layer comprises at least one pair of electrodes disposed on the sensor layer; and
at least one sensor coating disposed on at least one portion of the sensor layer and comprising an electrically active polymer, wherein the at least one intermediate path of the top layer extends from the entrance hole to the at least one sensor coating at least along the path length, such that the at least one intermediate path fluidically connects the entrance hole with the at least one sensor coating, and wherein the at least one sensor coating is electrically coupled to the at least one pair of electrodes on the sensor layer;
wherein the top layer is configured to allow a flow of the steam sterilant received from the entrance hole to the at least one sensor coating, and wherein the at least one sensor coating is configured to change an electrical impedance across the at least one pair of electrodes upon contact of the steam sterilant with the at least one sensor coating.

38-40. (canceled)
